(19) United States
(12) Patent Application Publication
(10) Pub. No.: US 2024/0065976 A1
(43) Pub. Date: Feb. 29, 2024

(54) A GASTRORETENTIVE DRUG DELIVERY SYSTEM

(71) Applicant: DUPONT NUTRITION USA, INC., Wilmington, DE (US)

(72) Inventors: Vinay Muley, New Delhi (IN); Rathnakar Palarapu, New Delhi (IN); Atul Ashok Kumar Hade, New Delhi (IN)

(21) Appl. No.: 18/261,284

(22) PCT Filed: Jan. 12, 2022
(86) PCT No.: PCT/EP2022/050524

§ 371 Date: Jul. 13, 2023

(30) Foreign Application Priority Data
Jan. 13, 2021 (IN) 202141001582

(51) Int. Cl. A61K 9/00; A61K 9/20; A61K 31/155; A61K 47/02; A61K 47/14; A61K 47/32; A61K 47/36; A61K 47/38

(52) U.S. Cl. CPC A61K 9/0065; A61K 9/0056; A61K 9/2009; A61K 9/2013; A61K 9/2018; A61K 9/205; A61K 9/2054; A61K 31/155; A61K 47/02; A61K 47/14; A61K 47/32; A61K 47/36; A61K 47/38

(57) ABSTRACT

A pharmaceutical or nutraceutical composition with raft-forming properties comprises
(a) sodium alginate,
(b) colloidal microcrystalline cellulose
(c) a bicarbonate, and
(d) a carbonate.

### FIELD OF INVENTION

The present invention relates to a composition with raft-forming properties comprising a sodium alginate and microcrystalline cellulose. The composition may be useful for the treatment of gastroesophageal disorders and as a gastro-retentive drug delivery system providing modulated release of an active ingredient.

### BACKGROUND OF THE INVENTION

Gastro-esophageal reflux disorders are a major source of discomfort world-wide and have been treated medically with antacids, H2 receptor antagonists and proton pump inhibitors as well as an anti-reflux composition containing an alginate and a bicarbonate/carbonate that forms a raft floating on top of the stomach contents when exposed to acid in the gastric juice upon ingestion. The raft prevents the stomach contents from passing into the esophagus and consequently protects the esophageal mucosa from irritation. Known raft-forming anti-reflux compositions that include sodium alginate, sodium bicarbonate and calcium carbonate include Gaviscon® and Gaviscon® Advance, both available from Reckitt Benckiser Healthcare.

Floating compositions have also been proposed as sustained release drug delivery systems due to their prolonged residence time in the stomach where they slowly release an active ingredient to the gastrointestinal environment ensuring an improved bioavailability of the active ingredient and reduced frequency of dosing. An overview of various floating drug delivery options is found in S. Sharma et al., International Journal of Research in Pharmaceutical and Biomedical Sciences 2(3), July-September 2011, pp. 954-958. One option is a gel-forming solution comprising sodium alginate or another gel-forming hydrocolloid and a bicarbonate or carbonate. On contact with gastric fluid, a viscous gel is formed containing entrapped bubbles of carbon dioxide that floats on top of the stomach contents.

To ensure a prolonged gastric residence time and good resilience, it is important to provide a composition that imparts advantageous properties to the raft formed in the stomach. Thus, an objective of the invention is to provide a gastro-retentive drug delivery system comprising sodium alginate as the gel-forming agent that exhibits improved raft strength and/or raft resilience.

### SUMMARY OF THE INVENTION

It has surprisingly been found that adding colloidal microcrystalline cellulose to a sodium alginate composition results in enhanced raft properties in terms of raft thickness, strength and/or resilience when subjected to in vitro testing that mimic conditions in the stomach.

Accordingly, the present invention relates to a pharmaceutical or nutraceutical composition with raft-forming properties, the composition comprising
(a) sodium alginate,
(b) colloidal microcrystalline cellulose
(c) a bicarbonate, and
(d) a carbonate.

In another aspect, the invention relates to a composition comprising components (a)-(d) for use in providing sustained or targeted release of a pharmaceutically or nutraceutically active ingredient.

### BRIEF DESCRIPTION OF THE DRAWINGS

FIG. 1 is a schematic depiction of a TA-XT2 device used to measure gel strength of a raft formed of the present composition in simulated gastric fluid.

FIGS. 2A and B show an experimental set-up for determining raft resilience, FIG. 2A showing the formation of a raft and FIG. 2B showing the tumble mixer in which the raft is subjected to mixing.

FIG. 3 schematically shows the steps of a process for preparing a composition of the invention.

### DETAILED DESCRIPTION OF THE INVENTION

### Definitions

In the present context the term “raft-forming properties” is intended to mean the ability of the inventive composition to make a floating barrier layer on top of the stomach contents when in contact with gastric acid.

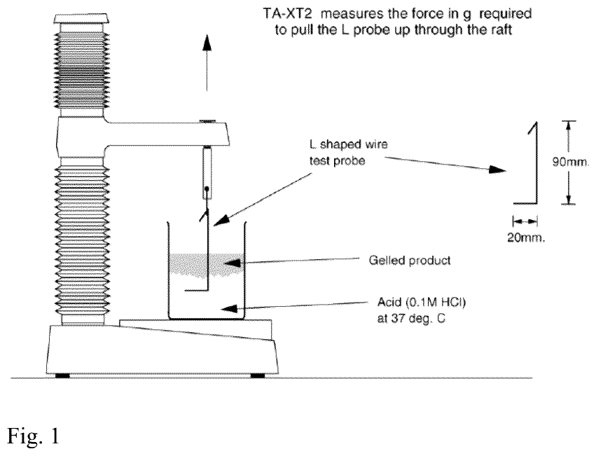

The term “raft strength” is determined as the force in g required to pull a probe up through a raft formed in simulated gastric fluid using the device shown in FIG. 1.

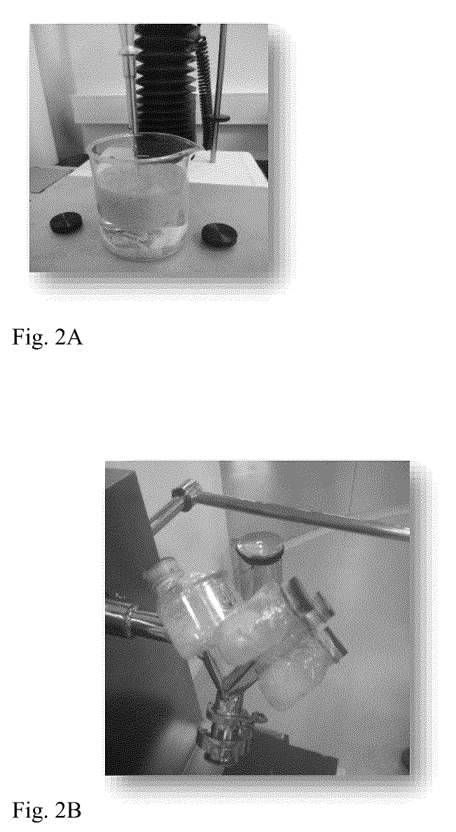

The term “raft resilience” is intended to mean the resistance of a raft to breaking up when subjected to physical movement in an experimental set-up such as when subjected to vigorous movement in a tumble mixer, cf FIG. 2B. Raft resilience is generally improved with increasing raft strength.

The term “colloidal microcrystalline cellulose” (colloidal MCC or cMCC in the following) is intended to mean MCC subjected to attrition in which the D₅₀ of the MCC particles is about 0.1-10 microns as measured by static light scattering in a particle size analyzer in which small particles scatter light at large angles and large particles scatter light at small angles, The scattering pattern produced by the cMCC sample is recorded and by applying Mie scattering theory the distribution of particle sizes can be calculated.

The term “D₅₀” as used in relation to particle size distribution denotes the diameter of the particle that 50% of a sample's volume is smaller than, and 50% of a sample's volume is larger than.

The term “flotation” is used to indicate the ability of a raft to float on top of the gastric fluid. Complete flotation is achieved when all insoluble material rises to the surface of the gastric fluid and is assessed as quick when taking place within about 1 minute of adding the composition to the gastric fluid.

The raft formed from the composition is indicated to be “coherent” when it is possible to remove the raft from the test gastric fluid in substantially one mass.

### EMBODIMENTS

Sodium alginate is included in the present composition as a gel forming agent.

Alginate is a family of linear binary copolymers of (1→4)-linked β-D-mannuronic acid (M) and α-L-guluronic acid (G) residues of widely varying composition and sequence. Work on the sequential structure of alginates reveals many fractions of widely differing composition: homopolymeric molecules of guluronic and mannuronic acid, nearly equal proportions of both monomers containing a large number of MG or GM dimer residues, just to name a few main fractions. Therefore, alginate is a true block copolymer composed of homopolymeric regions of M and G, termed M- and G-blocks, respectively, and interspersed with regions of alternating structure, MG- or GM-blocks.

Commercial alginates are produced mainly from a seaweed species selected from Laminaria hyperborea, Macrocystis pyrifera, Laminaria digitata, Ascophyllum nodosum, Laminaria japonica, Eclonia maxima, Lessonia nigrescens, and Durvillea Antarctica. In an industrial setting, gel strength under defined conditions is typically measured to estimate M-block/G-block ratio besides viscosity and pH on the specification sheet of alginate products.

It is preferred that the alginate is sodium alginate comprising at least 50% guluronic acid residues (G) more preferably 65-75% G. Such alginates have been found to form strong gels with divalent cations, e.g. Ca²⁺. Alginates with a G content of 65-75% may be extracted from L. hyperborea.

The sodium alginate is preferably one that has a low viscosity such as a viscosity in the range of 3-10 mPa·s as a 1% by weight aqueous solution at 20° C. measured by a Brookfield type RV viscometer using Brookfield spindle 2.

A particularly preferred sodium alginate to be included in the present composition is PROTANAL® LFR 5/60 which has a G content of 65-75% and a viscosity of 3.5-7 mPa·s as a 1% by weight aqueous solution at 20° C. measured by a Brookfield type RV viscometer using Brookfield spindle 2.

The sodium alginate may be present in a concentration of 25-50% by dry weight of the solids in the composition.

Microcrystalline cellulose (MCC) is a white, odorless, tasteless, relatively free flowing, crystalline powder that is virtually free from organic and inorganic contaminants. It is a purified, partially depolymerized cellulose manufactured by subjecting alpha cellulose obtained as a pulp from fibrous plant material to hydrolytic degradation typically with mineral acids. It is a highly crystalline particulate cellulose consisting primarily of crystalline aggregates which are obtained by removing amorphous regions (or paracrystalline regions) of a cellulosic fibril. MCC is used in a variety of applications including foods, nutraceuticals, pharmaceuticals and cosmetics.

Suitable starting materials for preparing the colloidal MCC include, for example, wood pulp such as bleached sulfite and sulfate pulps, corn husks, bagasse, straw, cotton, cotton linters, flax, hemp, ramie, fermented cellulose, etc. Microcrystalline cellulose may be produced by treating a source of cellulose, preferably alpha cellulose in the form of pulp from fibrous plant materials, with a mineral acid, preferably hydrochloric acid. The acid selectively attacks the less ordered regions of the cellulose polymer chain thereby exposing and freeing the crystalline sites which form crystallite aggregates which constitute the microcrystalline cellulose. These are then separated from the reaction mixture and washed to remove degraded by-products. The resulting wet mass, generally containing 40 to 75 percent moisture, is referred to in the art by several names, including hydrolyzed cellulose, hydrolyzed cellulose wetcake, level-off DP cellulose, microcrystalline cellulose wet cake or simply wetcake. Preferably, the aggregated MCC is acid hydrolyzed and 25-60% wt. in water.

When the wetcake is dried and freed of water the resulting product, microcrystalline cellulose, is a white, odorless, tasteless, relatively free-flowing powder, insoluble in water, organic solvents, dilute alkalis and acids. For a description of microcrystalline cellulose and its manufacture see U.S. Pat. No. 2,978,446.

For the present purpose, colloidal MCC may be prepared by subjecting hydrolyzed MCC aggregated crystallites, in the form of a high solids aqueous mixture, commonly known as “wetcake”, to an attrition process, e.g., extrusion, that substantially subdivides the aggregated cellulose crystallites into more finely divided crystallite particles. To prevent hornification, a protective hydrocolloid may be added before, during, or following attrition, but before drying. The protective hydrocolloid, wholly or partially, screens out the hydrogen bonds or other attractive forces between the smaller sized particles to provide a readily dispersible powder. Colloidal MCC will typically form stable suspensions with little to no settling of the dispersed solids. Carboxymethyl cellulose is a common hydrocolloid used for these purposes (see for example U.S. Pat. No. 3,539,365 (Durand et al.) and colloidal MCC products are for instance sold under the brand name AVICEL® by DuPont. A particularly favorable colloidal MCC for inclusion in the present composition is MCC co-attrited with CMC in a ratio of MCC to CMC in the range of from 85:15 to 90:10 (e.g. AVICEL® RC-591), and AVICEL® CE15 in which MCC is co-processed with guar gum. The latter MCC product is particularly suitable for formulating chewable tablets.

In the present composition, colloidal MCC acts as a stabilizer and suspending agent.

The colloidal MCC may be present in a concentration of 8-20% by dry weight of the solids in the composition.

The present composition further comprises a bicarbonate and a carbonate in amounts that generate sufficient quantities of carbon dioxide on contact with gastric acid for the composition to float on top of the stomach contents. The bicarbonate is preferably present in a concentration of 15-30%, more preferably 18-25%, by dry weight of the solids in the composition. The bicarbonate may be selected from potassium or sodium bicarbonate, preferably sodium bicarbonate.

The carbonate is preferably present in a concentration of 8-20% by dry weight of the solids in the composition. The carbonate may be selected from calcium, magnesium or aluminium carbonate, preferably calcium carbonate. Calcium ions are released in gastric acid and interact with sodium alginate to form calcium alginate which is insoluble in water, thereby contributing to raft formation and increased raft strength.

It has surprisingly been found that raft properties of the composition may be modified by adding a polyethylene oxide with a high molecular weight (100,000-7,000,000 daltons). Addition of high molecular weight polyethylene oxide to a composition of the invention comprising sodium alginate and colloidal MCC co-processed with CMC results in increased raft strength and resilience, cf. Example 2 below. The high molecular weight polyethylene oxide also acts as a suspending agent for water-insoluble components in the composition, such as colloidal MCC and water-insoluble active ingredients. An example of polyethylene oxide suitable for the present purpose is POLYOX™ Sentry, available from DuPont. The polyethylene oxide may be present in a concentration of 1-10% by dry weight of the solids in the composition.

The raft properties of the present composition may also be modified by adding a hydroxypropyl methylcellulose (HPMC). HPMC is a cellulose ether that is used as a thickener in aqueous compositions and increases the viscosity thereof. The viscosity increase depends on the molecular weight and concentration of the HPMC in water. HPMC may also act as a suspending agent for water-insoluble components in the composition. Examples of suitable HPMCs are METHOCEL™ K grades, in particular METHOCEL™ K100M which is a HPMC with a methoxyl substitution of 19.0-24.4%, a hydroxypropoxyl substitution of 7.0-12.0% and a viscosity of 75,000-140,000 mPa·s determined as a 2% solution in water at 20° C. in an Ubbelohde viscometer according to ASTM 2363-79 (Reapproved 2006). METHOCEL^{YM} K grade HPMCs are commercially available from DuPont. The HPMC may be present in a concentration of 1.5-10% by dry weight of the solids in the composition.

The present composition may either be used as such to provide a treatment of gastro-esophageal reflux disorders or as a gastro-retentive drug delivery system (GRDDS) to provide sustained or targeted release of a pharmaceutically or nutraceutically active ingredient. The GRDDS may suitably be in the form of an aqueous suspension or a capsule, tablet, powder or granules, preferably in the form of an aqueous suspension or chewable tablet. Examples of active ingredients that may favourably be administered as a GRDDS are dietary supplements such as curcumin, a vitamin, such as vitamin D or folate, a prebiotic or probiotic, and minerals such as magnesium, zinc or iron, or an active pharmaceutical ingredient selected from antidiabetics such as metformin hydrochloride; antibiotics such as erythromycin, cephalosporin, minocycline, amoxicillin and ciprofloxacin; analgesics and anti-inflammatory agents such as acetaminophen, ibuprofen, ketoprofen, indomethacin or naproxen; antacids such as aluminium hydroxide and magnesium hydroxide; H₂ receptor antagonists such as ranitidine, cimetidine and famotidine; antihistamines such as chlorpheniramine maleate, diphenhydramine hydrochloride and triprolidine hydrochloride; acidic drugs and the very weakly basic drugs such as salicylic acid, aspirin, thiopental, secobarbital and antipyrine etc.

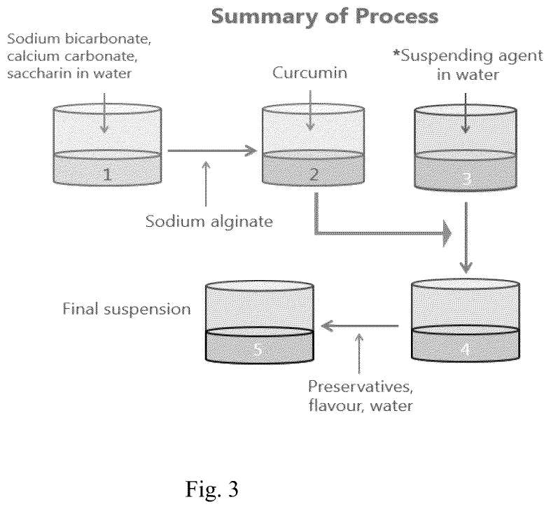

The present composition may be prepared as outlined in FIG. 3 by the steps of: dispersing bicarbonate and carbonate in water and optionally water-soluble components and/or active ingredients in a first vessel and adding sodium alginate with mixing;
dispersing and activating colloidal MCC in water in a second vessel;
adding the bicarbonate, carbonate and sodium alginate dispersion from the first vessel to the dispersion of colloidal MCC from the second vessel; and
optionally adding preservatives and flavour and completing the volume with water.

To make solid dosage forms such as tablets the required ingredients are blended in a suitable tumbler mixer and compressing the tablets to the desired size.

The invention is further described in the following examples.

### EXAMPLES

### Methods

Raft resilience was determined according to F. C. Hampson et al., International Journal of Pharmaceutics 294, 2005, pp. 137-147, by forming rafts in 200 ml glass bottles by adding the inventive composition as an aqueous suspension to 150 ml of 0.1M HCl, previously equilibrated at 37° C. Rafts were developed for 30 min (FIG. 2A), then the bottles were capped and placed in a tumble mixer (Tied to a V-blender) which is set to rotate at 20 rpm, to simulate gastric agitation as shown in FIG. 2B. The rafts were assessed visually for thickness, coherence, intactness after total periods of agitation of 2, 5, 10, 20, 30, 45, and to a maximum of 60 min, or until such time that a raft could no longer be detected. A raft was defined, for visual assessment, as two or more floating gels at least 15 mm in diameter. Raft resilience was defined as the last time point at which a raft was observed.

Raft strength was determined according to F. C. Hampson, supra, by adding the inventive composition to 150 ml of 0.1 M HCl maintained at 37° C. in a 250 ml glass beaker. Each raft was formed around an L-shaped stainless steel wire probe held upright in the beaker throughout the entire period of raft development. After 30 min. of raft development, the beaker was placed on the table of a TA-XT Texture Analyser (Stable Micro Systems, UK), the wire probe was hooked onto the Texture Analyser arm and pulled vertically up through the raft at a rate of 5 mm per second. The force (g) required to pull the wire probe up through the raft was recorded by the Texture Analyser. A schematic depiction of the TA-XT device is shown in FIG. 1.

### Example 1

### Aqueous Suspension of Sodium Alginate and Colloidal MCC

An aqueous suspension was prepared from the following ingredients as outlined in FIG. 3.

| | Amount | Solids | Solids |
|---|---|---|---|
| Ingredient | (g) | % w/v | % w/w |
| Sodium alginate Protanal ® LFR 5/60 | 100 | 5 | 37.68 |
| Potassium bicarbonate | 53.4 | 2.67 | 20.12 |
| Calcium carbonate | 48 | 2.4 | 18.09 |
| Avicel RC 591 | 30 | 1.5 | 11.30 |
| Sodium hydroxide (for pH adjustment) | 6.0 | 0.3 | 2.26 |
| (10% solution) | | | |
| Methyl (E218) parahydroxybenzoate | 8.0 | 0.4 | 3.01 |
| (or sodium salt) | | | |
| Propyl (E216) parahydroxybenzoate | 1.2 | 0.6 | 4.52 |
| (or sodium salt) | | | |
| Sodium saccharin | 4.0 | 0.2 | 1.51 |
| Peppermint flavor | 4.0 | 0.2 | 1.51 |
| Total Solids | | 13.27 | 100.00 |
| DI water | Qs 2000 mL | | |

When tested according to the methods discussed above, the composition was found to have the following raft properties

| S. No. | Test Parameter | Results | |
|---|---|---|---|
| 1 | Raft strength | 19 | g |
| 2 | Viscosity | 2 | cps |
| 3 | Raft Resilience | Up to 30 | min |
| 4 | Raft Thickness | 32 | mm |
| 5 | Raft Flotation | LT 1 | min |

| | | |
|---|---|---|
| 6 | Raft Coherence | Good |

These results were compared to raft properties obtained with Comparative Example A (a commercial composition (Gaviscon® Advance)) under similar conditions. Comparative Example A includes the following ingredients.

| | Amount | Solids | Solids |
|---|---|---|---|
| Ingredient | (g) | % | % w/w |
| Sodium alginate Protanal ® LFR 5/60 | 200 | 10 | 42.27 |
| Potassium bicarbonate | 40 | 2 | 22.57 |
| Calcium carbonate | 40 | 2 | 13.52 |
| Carbomer (Carbopol 974P or 971P) | 13.0 | 0.65 | 12.68 |
| Sodium hydroxide (for pH adjustment) | 6.0 | 0.3 | 1.69 |
| (10% solution) | | | |
| Methyl (E218) parahydroxybenzoate | 8.0 | 0.4 | 3.38 |
| (or sodium salt) | | | |
| Propyl (E216) parahydroxybenzoate | 1.2 | 0.006 | 0.51 |
| (or sodium salt) | | | |
| Sodium saccharin | 4.0 | 0.2 | 1.69 |
| Peppermint flavor | 4.0 | 0.2 | 1.69 |
| Total solids | 316.2 | 11.83 | 100 |
| DI water | Qs 2000 mL | NA | NA |
| Grand Total | 2316.2 | 11.83 | 100 |

When tested according to the methods discussed above, the composition was found to have the following raft properties.

| S. No. | Test Parameter | Results | |
|---|---|---|---|
| 1 | Raft strength | 16 | g |
| 2 | Viscosity | 6 | cps |
| 3 | Raft Resilience | Up to 45 | min |
| 4 | Raft Thickness | 20 | mm |
| 5 | Raft Flotation | LT 1 | min |

| | | |
|---|---|---|
| 6 | Raft Coherence | Good |

It appears from the results that the inventive composition of Example 1 containing colloidal MCC is superior to Comparative Example A in terms of raft strength and raft thickness, while raft resilience is somewhat higher in Comparative Example A.

### Example 2

### Aqueous Suspension of Sodium Alginate, Colloidal MCC and Polyethylene Oxide

An aqueous suspension was prepared from the following ingredients as outlined in FIG. 3.

| | Amount | Solids | Solids |
|---|---|---|---|
| Ingredient | (g) | % w/v | % w/w |
| Sodium alginate Protanal ® LFR 5/60 | 25 | 5 | 42.27 |
| Sodium bicarbonate | 13.35 | 2.67 | 22.57 |
| Calcium carbonate | 8.0 | 1.6 | 13.52 |
| Avicel RC 591 | 7.5 | 1.5 | 12.68 |
| Polyox Sentry | 1 | 0.2 | 1.69 |
| Methyl (E218) parahydroxybenzoate | 2.0 | 0.4 | 3.38 |
| (or sodium salt) | | | |
| Propyl (E216) parahydroxybenzoate | 0.3 | 0.06 | 0.51 |
| (or sodium salt) | | | |
| Sodium saccharin | 1.0 | 0.2 | 1.69 |
| Peppermint flavor | 1.0 | 0.2 | 1.69 |
| Total solids | | 11.83 | 100.00 |
| DI water | Qs 500 mL | NA | NA |
| Total | 559.15 | 11.83 | 100 |

When tested according to the methods discussed above, the composition was found to have the following raft properties.

| S. No. | Test Parameters | Results | |
|---|---|---|---|
| 1 | Raft strength | 17 | g |
| 3 | Raft Resilience | 60 | min |
| 4 | Raft Weight | 53.7 | g |
| 5 | Raft Thickness | 30.4 | mm |

| | | |
|---|---|---|
| 6 | Raft Flotation | Quick & Good |

It appears from the results that the inventive composition of Example 2 containing both colloidal MCC and polyethylene oxide is superior to Comparative Example A in terms of raft strength, raft resilience and raft thickness.

### Example 3

### Chewable Tablets Containing Sodium Alginate, Colloidal MCC and Optionally Polyethylene Oxide

Chewable tablet containing the following ingredients were prepared.

| | With | Without |
|---|---|---|
| Ingredient | Polyox | Polyox |
| Sodium alginate Protanal ® LFR 5/60 | 1000 | 1000 |
| Sodium bicarbonate | 500 | 500 |
| Calcium carbonate | 300 | 300 |
| Mannitol | 200 | 200 |
| Avicel CE15 | 200 | 200 |
| Polyox Sentry | 200 | 0 |
| Alubra (sodium stearyl fumarate) | 100 | 100 |
| Total | 2500 mg | 2300 mg |

When tested according to the method discussed above, the tablet formulations were found to have the following raft properties.

| | | With | Without |
|---|---|---|---|
| S. No. | Test Parameters | Polyox | Polyox |
| 1 | Raft strength | 6.6 g | 12.9 g |
| 4 | Raft Weight (gm) | 50.4 | 59.2 |
| 5 | Raft Thickness (mm) | 26.05 | 27.01 |
| 6 | Flotation | Quick | Quick |

Although the observed raft strength was lower in compositions containing with Polyox, the suspension stability was found to be good along with quick flotation and good raft thickness and weight.

### Example 4

### An Aqueous Suspension of Sodium Alginate and Colloidal MCC Containing Metformin HCl

An aqueous suspension was prepared from the following ingredients as outlined in FIG. 3.

| | Amount | Solids | Solids |
|---|---|---|---|
| Ingredient | (g) | % w/v | % w/v |
| Metformin Hydrochloride | 12.5 | 2.08 | 17.91 |
| Sodium alginate Protanal ® LFR 5/60 | 25 | 4.17 | 35.82 |
| Sodium bicarbonate | 13.50 | 2.25 | 19.34 |
| Calcium carbonate | 8.0 | 1.33 | 11.46 |
| Avicel RC 591 | 7.5 | 1.25 | 10.74 |
| Methyl (E218) parahydroxybenzoate | 2.0 | 0.33 | 2.87 |
| (or sodium salt) | | | |
| Propyl (E216) parahydroxybenzoate | 0.3 | 0.05 | 0.43 |
| (or sodium salt) | | | |
| Sodium saccharin | 1.0 | 0.17 | 1.43 |
| Total Solids | 69.8 | 11.63 | 100.00 |
| DI Water | 530.2 | NA | NA |
| Total | 600 | 11.63 | 100 |

When tested according to the methods discussed above, the aqueous suspension of Example 4 was found to have the following raft properties.

| | Raft Characteristics | Value | |
|---|---|---|---|
| 1 | Raft strength | 17.83 | g |
| 2 | Raft Resilience | 90 | min |

| | | |
|---|---|---|
| 3 | Flotation | Good & Quick |

These results were compared to raft properties obtained with Comparative Example B (a commercial composition (Gaviscon® Advance) containing metformin HCl) under similar conditions. Comparative Example B includes the following ingredients.

| | Amount | Solids | Solids |
|---|---|---|---|
| Ingredient | (g) | % w/v | % w/w |
| Metformin Hydrochloride | 12.5 | 2.08 | 19.11 |
| Sodium alginate Protanal ® | 25 | 4.17 | 38.23 |
| LFR 5/60 | | | |
| Sodium bicarbonate | 13.35 | 2.23 | 20.41 |
| Calcium carbonate | 8.0 | 1.34 | 12.23 |
| Carbomer (Carbopol 974P or 971P) | 3.25 | 0.54 | 4.97 |
| Methyl (E218) parahydroxybenzoate | 2.0 | 0.33 | 3.06 |
| (or sodium salt) | | | |
| Propyl (E216) parahydroxybenzoate | 0.3 | 0.05 | 0.46 |
| (or sodium salt) | | | |
| Sodium saccharin | 1.0 | 0.17 | 1.53 |
| Total Solids | 65.4 | 10.91 | 100 |
| DI Water | 533.1 | NA | NA |
| Grand Total | 598.5 | 10.91 | 100 |

When tested according to the methods discussed above, the aqueous suspension of Comparative Example B was found to have the following raft properties.

| Raft Characteristics | Value | |
|---|---|---|
| Raft strength | 15.70 | g |
| Raft Resilience | 60 | min |

| | |
|---|---|
| Flotation | Good & Quick |

It appears from the results that the inventive composition of Example 4 containing colloidal MCC is superior to Comparative Example B in terms of raft strength and raft resilience.

1. A pharmaceutical or nutraceutical composition with raft-forming properties, the composition comprising
(a) sodium alginate,
(b) colloidal microcrystalline cellulose,
(c) a bicarbonate, and
(d) a carbonate

2. The composition of claim 1, wherein the sodium alginate has a viscosity in the range of 3-10 mPa·s as a 1% by weight aqueous solution at 20° C. measured by a Brookfield type RV viscometer using Brookfield spindle 2.

3. The composition of claim 1, wherein the sodium alginate has a guluronic acid content of ≥50%.

4. The composition of claim 1, wherein the concentration of sodium alginate is in the range of 25-50% by dry weight of the solids in the composition.

5. The composition of claim 1, wherein the colloidal microcrystalline cellulose is one that is co-processed with carboxymethylcellulose or guar gum.

6. The composition of claim 5, wherein the co-processed colloidal microcrystalline cellulose has a ratio of microcrystalline cellulose to carboxymethylcellulose in the range of 85:15 to 90:10.

7. The composition of claim 1, wherein the concentration of colloidal microcrystalline cellulose is in the range of 8-20% by dry weight of the solids in the composition.

8. The composition of claim 1, wherein the bicarbonate is sodium bicarbonate.

9. The composition of claim 1, wherein the concentration of the bicarbonate is in the range of 15-30% by dry weight of the solids in the composition.

10. The composition of claim 1, wherein the carbonate is calcium carbonate.

11. The composition of claim 1, wherein the concentration of the carbonate is 8-20% by dry weight of the solids in the composition.

12. The composition of claim 1, further comprising polyethylene oxide with a molecular weight of 100,000-7,000,000 daltons.

13. The composition of claim 12, wherein the concentration of polyethylene oxide is in the range of 1-10% by dry weight of the solids in the composition.

14. The composition of claim 1, further comprising hydroxypropyl methylcellulose.

15. The composition of claim 14, wherein the concentration of hydroxypropyl methylcellulose is 1.5-10% by dry weight of the solids in the composition.

16. The composition of claim 1, which is in the form of an aqueous suspension or a capsule, tablet, powder or granules.

17. The composition of claim 1, further comprising a pharmaceutically or nutraceutically active ingredient.

18. The composition of claim 1, for use in providing sustained or targeted release of a pharmaceutically or nutraceutically active ingredient.

19. The composition of claim 1, wherein the sodium alginate has a guluronic acid content of 65-75%.

20. The composition of claim 1, which is in the form of an aqueous suspension or chewable tablet.
